(19) United States
(12) Patent Application Publication
(10) Pub. No.: US 2009/0228081 A1
(43) Pub. Date: Sep. 10, 2009

(54) MOUTH PIECE FOR UV-LIGHT DELIVERY

(76) Inventor: Thomas Perez, Chicago, IL (US)

Correspondence Address: ADAM K. SACHAROFF;MUCH SHELIST DENENBERG AMENT & RUBENSTEIN, 191 N. WACKER DRIVE, Suite 1800, CHICAGO, IL, 60606-1615, US

(21) Appl. No.: 12/468,621

(22) Filed: May 19, 2009

Related U.S. Application Data
(63) Continuation-in-part of application No. 11/140,272, filed on May 27, 2005.

(51) Int. Cl. A61N 5/06

(52) U.S. Cl. 607/94

(57) ABSTRACT

In one embodiment there is provided a system for delivering UV-light under a patient's tongue. The system includes a UV-light source in communication with a pair of tubes and a U-shaped double trough mouth piece with upper and lower troughs adapted to engage upper and lower teeth. The upper and lower troughs being interconnected by a relatively flat base surface. A pair of windowed apertures are centrally located in the double trough region and extend from the front to the rear of the double trough region. Each windowed aperture includes a pair of openings extending out through the lower base surface to accommodate the pair of tubes, such that the tips of the tubes are capable of extending through the openings and exiting through the lower base surface.

### CROSS REFERENCE TO RELATED APPLICATIONS

This application is a continuation in part of U.S. application serial No. 11/140,272 filed May 27, 2005.

### BACKGROUND OF THE INVENTION

1. Field of the Invention

The present invention relates to a method and apparatus for providing light to blood.

2. Background of the Prior Art

Ultraviolet (UV) light can be used to treat a multitude of medical problems, including for example bacterial, viral and fungal infections, poisoning, fatigue, Alzheimer's disease, allergies and asthma, rheumatic diseases and arthritis, diabetes, hepatitis, and cancer. UV light sterilizes the blood and acts as an antibiotic.

The UV light is applied either to the patient's skin or directly to the blood. If the UV light is applied to the skin it is typically provided to the patient's skin either with a wrap or lamp.

UV light is commonly used to treat jaundiced babies. Because infant's skin is thin and the blood vessels are close to the surface, UV light is typical applied to the skin when treating jaundiced babies.

Applying the UV light directly to a patient's blood supply is known as photoluminescence or UV blood illumination (UBI). UV blood illumination increases oxygen, destroys toxins and boosts the immune system.

In prior art UBI, a small amount of blood is drawn from the patient, up to about 250 cc. The body has about 5.6 L of blood. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for a method of providing UV light to a patient's entire blood supply, not just a small portion of it. There is a need for a system that is convenient for the patient, which does not require regular doctor visits. There is a need for a simple system that can be used by the patient in his home.

### SUMMARY OF THE INVENTION

The present invention is a light device or a portable light pack that irradiates the mucous membrane under the tongue. The light pack has a battery or other power supply and a light source. The light source emits light at one or more therapeutic wavelengths. Preferably, the light is UV light at one or more therapeutic wavelegths. The UV light source is typically LEDs that emit UV-A or UV-C light or a combination of UV-A and UV-C light. The light pack or device is inserted into the patient's mouth, preferably under the tongue. Fiber optic strand(s) run through a tube to illuminate the mucous membrane under the tongue. Capillaries are plentiful and close to the surface under the tongue, thus illuminating the blood. The present invention uses a mouth-guard specifically designed an used to administer and position the UV light to the mucous membrane under the tongue.

In a first embodiment there is provided a system for delivering UV-light under a patient's tongue. The system includes providing for a UV-light source in communication with a pair of tubes for transmitting the UV-light from the source to end tips defined by the pair of tubes separately. The system also includes a mouth piece having a U-shaped double trough region defined as an upper trough and a lower trough adapted to engage upper and lower teeth of a patient, respectively. The upper trough and lower trough being interconnected by a relatively flat base surface. The base surface provides for an upper base surface for the upper trough and a lower base surface for the lower trough. The mouth piece further includes a pair of windowed apertures centrally located in the double trough region and extending from a front portion to a rear portion of the double trough region. Each windowed aperture includes a pair of openings extending out through the lower base surface. The pair of openings are sized to accommodate the pair of tubes such that the tips of the tubes are capable of extending through the openings and exiting through the lower base surface.

The system may further define the upper trough as including an upper outer peripheral wall member and an upper inner peripheral wall member laterally spaced from the upper outer peripheral wall member, both the upper outer and upper inner peripheral wall members being interconnected with the upper base surface. And may further define the lower trough as including a lower outer peripheral wall member and a lower inner peripheral wall member laterally spaced from the lower outer peripheral wall member, both the lower outer and lower inner peripheral wall members being interconnected with the lower base surface.

In addition, the upper outer peripheral wall member may have a defined height higher than a defined height of the upper inner peripheral wall member. The system may further include indents positioned midway of the U-shaped upper trough at either or both the upper and lower outer peripheral wall members.

In other embodiments the pair of tubes further includes an indented region sized to fit within the pair of openings in the windowed apertures or the pair of tubes includes an enlarged region at a distance away from the tip, the enlarged region having a diameter larger then a diameter of an opening defined by the pair of openings.

In yet other embodiments, the invention may provide for a blood illuminator, defined to include a light source adapted to emit UV-C light and a power supply for supplying power to the light source; a pair of light guides, each light guide having a first end for receiving the UV-C light from the light source and having a second end adapted to emit the UV-C light; and a mouth piece having a pair of bored apertures separately corresponding to the pair of light guides, each bored apertures having an entrance near a front portion of the mouth piece and an exit near a bottom portion of the mouth piece, wherein the pair of light guides are inserted through the pair of board apertures, such that the second ends of the pair of light guides may extend outwardly from the bottom portion of the mouthpiece.

And in yet other embodiments, there is provided for a blood illuminator, defined to include a light source adapted to emit UV-C light and a power supply for supplying power to the light source; a pair of light guides, each light guide having a first end for receiving the UV-C light from the light source and having a second end adapted to emit the UV-C light; and a mouth piece having a pair of bored apertures separately corresponding to the pair of light guides, each bored apertures having an entrance near a front portion of the mouth piece and an exit near a bottom portion of the mouthpiece, wherein the pair of light guides are inserted through the pair of board apertures, such that the second ends of the pair of light guides may extend outwardly from the bottom portion of the mouth piece and wherein the mouth piece includes a grove along a center region of the front portion.

Still further, there may be provided for a method of illuminating blood with light. The method including the following steps, providing a light source adapted to emit UV-C light; providing a pair of light guides, each light guide having a first end for receiving the UV-C light from the light source and having a second end adapted to emit the UV-C light; placing a mouthpiece in a mouth of a patient, the mouthpiece having a pair of bored apertures separately corresponding to the pair of light guides, each bored apertures having an entrance near a front portion of the mouthpiece and an exit near a bottom portion of the mouthpiece, wherein the pair of light guides are inserted through the pair of board apertures, such that the second ends of the pair of light guides may extend outwardly from the bottom portion of the mouthpiece and towards a bottom region of the mouth of the patient, and wherein the mouthpiece includes a grove along a center region of the front portion to accommodate a labial frenulum defined in the mouth of the patient; and supplying power to the light source such that the bottom region of the mouth of the patient is illuminated, wherein the UV-C light penetrates capillaries in the mouth to allow the UV-C light to illuminate blood of the patient.

Numerous advantages and features of the invention will become readily apparent from the following detailed description of the invention and the embodiments thereof, and from the accompanying drawings.

### BRIEF DESCRIPTION OF THE DRAWINGS

A fuller understanding of the foregoing may be had by reference to the accompanying drawings, wherein:

FIG. 1A is a portable light pack;

FIG. 1B is a sub-lingual light irradiation delivery device inserted into a patient's mouth

FIG. 2 is a sub-lingual light irradiation delivery device in accordance with one embodiment of the present invention;

FIG. 3 is a light guide for use with the light pack and delivery device or mouth guard;

FIG. 4 is an enlarged view of the tip of the light guide from FIG. 3;

FIG. 5A is a front perspective view of a mouth guard in accordance with a second embodiment of the present invention;

FIG. 5B is the mouth guard from FIG. 5A illustrating the light guides before insertion through the mouth guard;

FIG. 5C is the mouth guard from FIG. 5A illustrating the light guides inserted through the mouth guard;

FIG. 6 is the mouth guard from FIG. 5A showing the mouth guard prior to being inserted into a patient's mouth;

FIG. 7A is the mouth guard from FIG. 5A illustrating the mouth guard from a bottom perspective view;

FIG. 7B is the mouth guard from FIG. 7A illustrating the light guides prior to insertion through the mouth guard;

FIG. 7C is the mouth guard from FIG. 7A illustrating the light guides inserted through the mouth guard;

FIG. 8 is a side view of the mouth guard from FIG. 7C; and

FIG. 9 is a back view of the mouth guard from FIG. 5A.

### DETAILED DESCRIPTION OF THE INVENTION

While the invention is susceptible to embodiments in many different forms, there are shown in the drawings and will be described herein, in detail, the preferred embodiments of the present invention. It should be understood, however, that the present disclosure is to be considered an exemplification of the principles of the invention and is not intended to limit the spirit or scope of the claims by the embodiments illustrated.

Light at one or more therapeutic wavelength, such as ultraviolet light (UV), is used to treat many diseases including infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Preferably, UV light at one or more therapeutic wavelength is utilized in the present invention. More preferably the light is either UV-A or UV-C light is utilized in the present invention. For some conditions and/or diseases UV-A light is more effective than UV-C and for other conditions and/or diseases UV-C light is more effective than UV-A light. The wavelengths or wavelengths of light to be used to treat the patient are selected based on the wavelength or wavelength that will best treat the condition or disease of the patient.

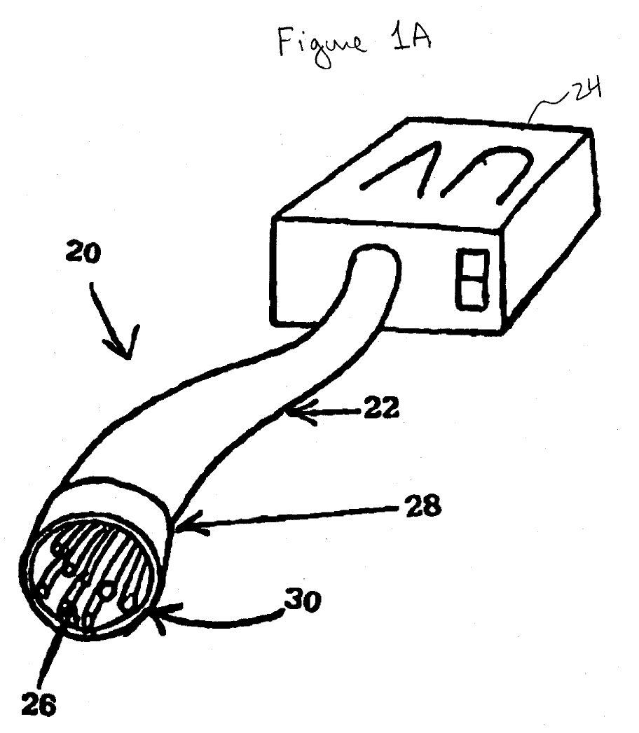

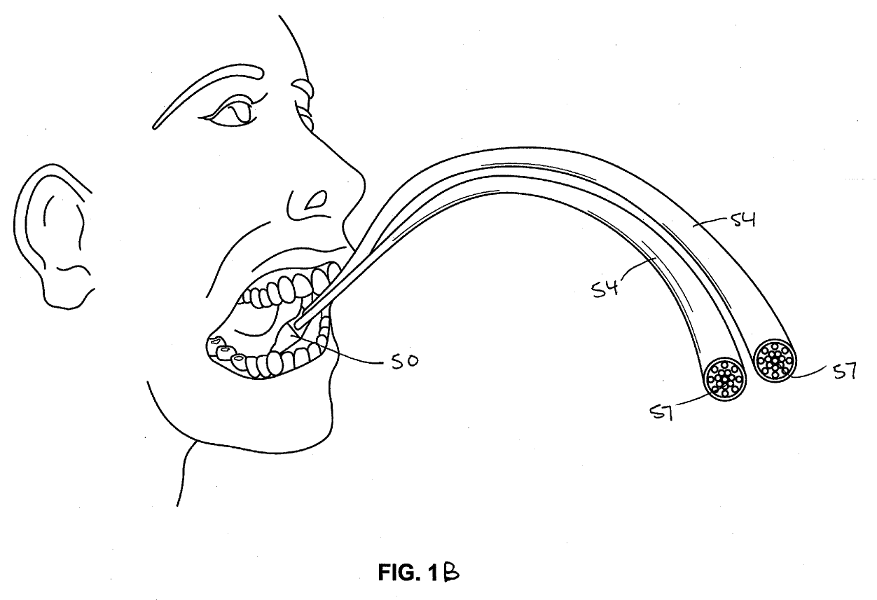

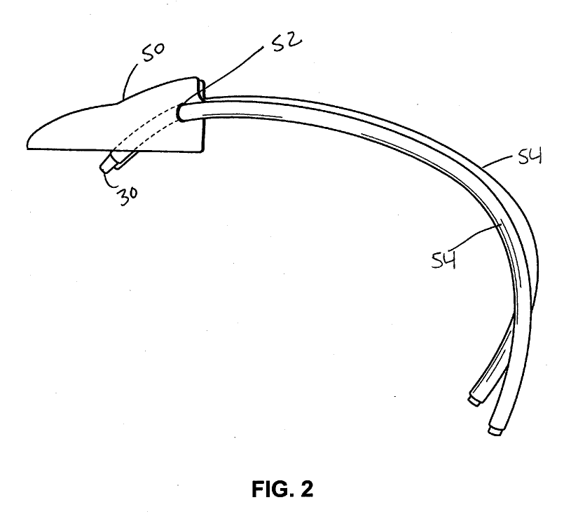

In one embodiment, FIGS. 1A-2, there is provided a light device enclosed within a portable light pack 20 that directly supply light at a therapeutic wavelength(s), preferably UV light, to the patient's blood supply. Portable light pack 20 comprises housing 24, battery or other power source (not shown) and light source (not shown). Preferably, the light source (not shown) is LEDs (not shown) or a light guide transferring the light from a source bulb, medical grade UV light bulb, or similar source that emits UV light at a therapeutic wavelength(s). More preferably, the light is UV-C light, UV-A, UV-B light or a combination thereof. Alternatively, a plurality of LEDs, having one or more different wavelengths of light at one or more therapeutic wavelengths is used. It is preferable that a substantial portion of the emitted light be UV-C and/or UV-A.

In one embodiment, light pack 20 has a light guide 22 made of rubber or other flexible tubing for housing one or more fiber optic strands 26. Alternatively, a liquid core light guide or other known light guide can be used. Emitted radiation travels to the end tip 30 of the light guide 22 and is emitted outwardly therefrom. Emitted radiation directly illuminates patient's blood. Light guide 22 has transparent cover 28 at the end.

The light pack 20 allows therapeutic wavelength(s) of light to be supplied directly to the blood. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence.

Light pack 20 could be turned on and off manually. Alternatively, light pack 20 could automatically turn off the light source after a set treatment time, such as 20 minutes. Light pack 20 could have a controller such as a computer or other smart interface that limits the number of treatments given time period, limits the total amount of treatment time in a given time period, automatically provides treatments, pulses the light source, or provides only particular wavelengths. The computer or other smart interface could keep a treatment record. The computer or other smart interface could communicate wirelessly, via the Internet or through other electronic means to automatically update the doctor's treatment records. Computer preferably can automatically adjust treatment time, wavelength or other factors based on patient input, doctor orders or other data.

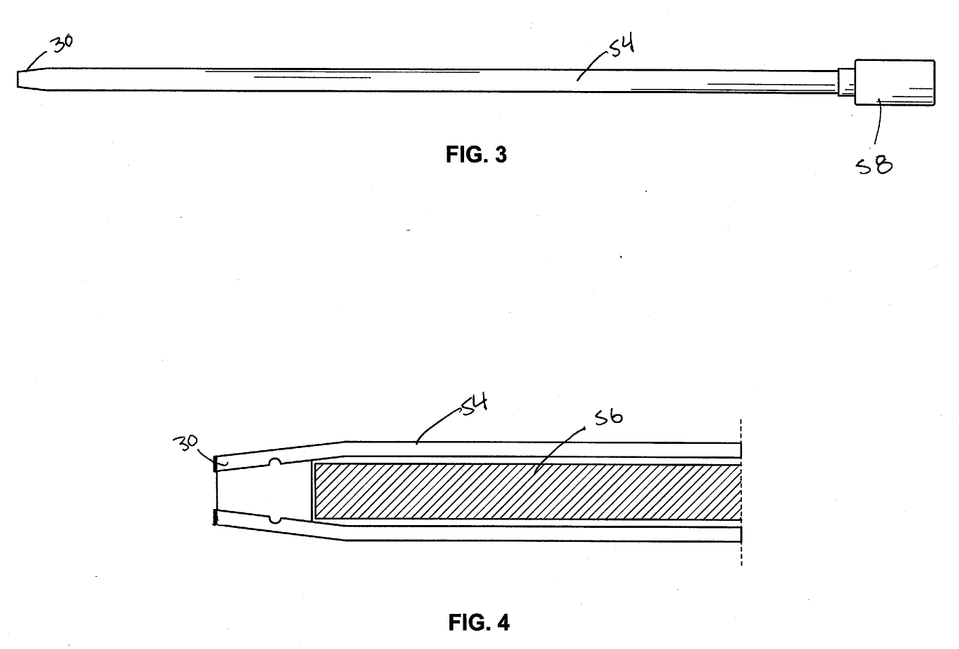

Continuing to refer to FIGS. 1A through 4, light at a therapeutic wavelength is administered under the tongue. The capillaries under the tongue are close to the surface. These capillaries are very sensitive. Capillary exposure of the mucus membrane is significantly greater than other exposed body surfaces. The greater capillary exposure allows for greater penetration of the ultraviolet spectrum. Light device 20 comprises a mouthpiece 50 for holding and aligning the light source under the tongue. Mouthpiece 50 is inserted into patient's mouth under the tongue. The mouthpiece 50 has at least one aperture 52 through which tubing 54 is inserted. Tubing 54 is preferably plastic tubing and is preferably flexible. The tubing may be adjustably inserted through the at least one aperture 52 to allow for individual adjustment by the patient or doctor to a preferred treatment location under the patient's tongue. Alternatively, tubing 54 can be mounted in the preferred position such that each time the mouthpiece 50 is used, the light is administered at the same location. Preferably, mouthpiece 50 is molded to the shape of patient's 10 mouth.

There may be fused silica fiber optic bundles 57 in the tubing 54. Fused silica fiber optic bundles 54 may be used because they do not emit any heat. The fiber optic bundles deliver UV light at a therapeutic wavelength sublingually. The light is preferably UV-A, UV-C or a combination thereof. In yet another alternative, light source is a LED or small light bulb at the end of the flexible tube adapted to directly irradiate the mucus membrane under the tongue. This delivery system is preferred for relatively young patients without a life threatening disease. In yet another alternative, the tubing 54 may be housing a liquid light guide core 56. In addition, the tubing 54 may also include an attachment end 58 used to secure the tubing 54 to light pack housing 24 or other suitable light emitting source.

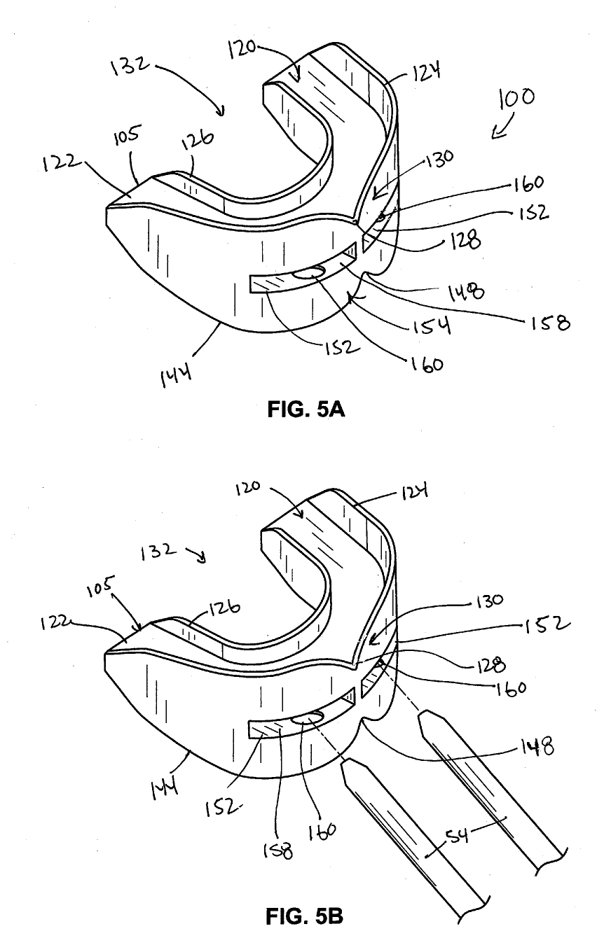

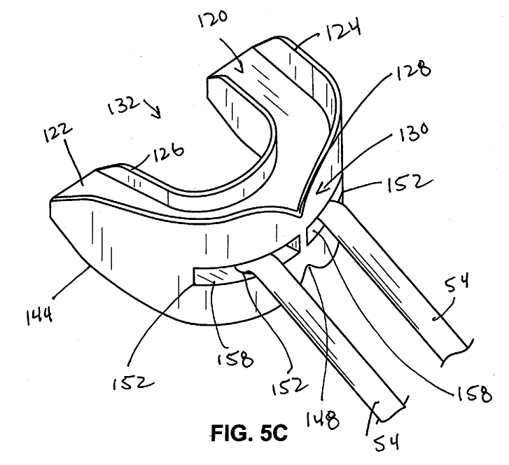

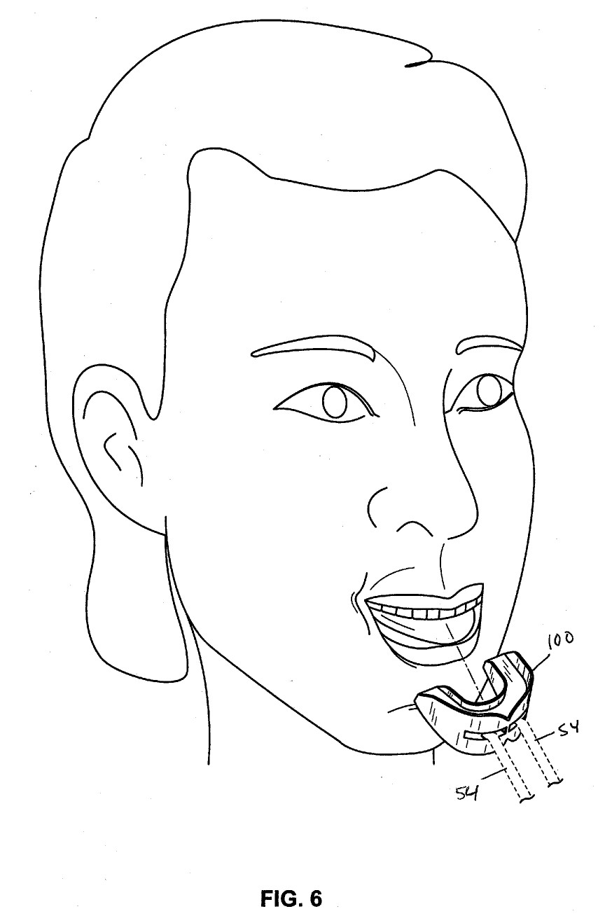

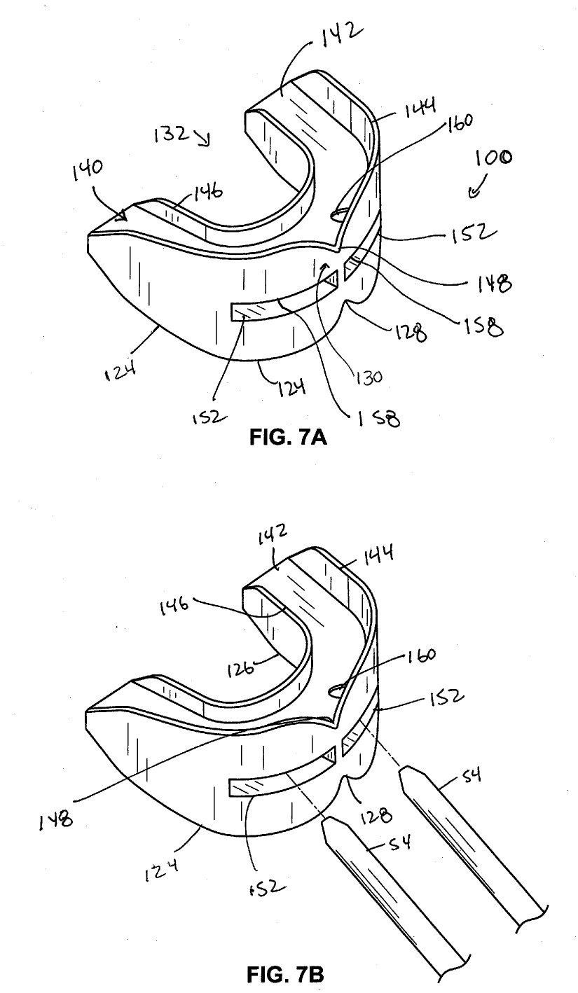

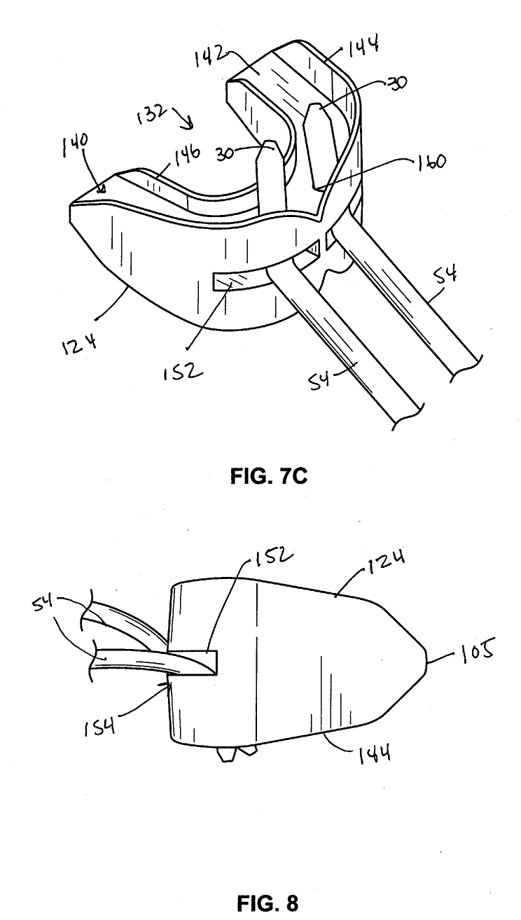

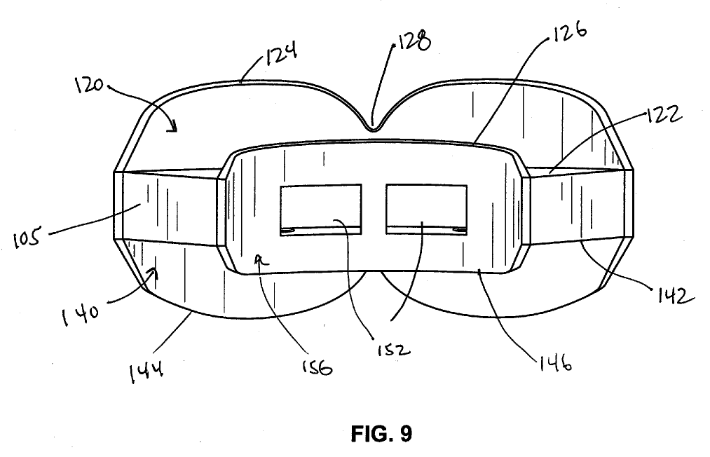

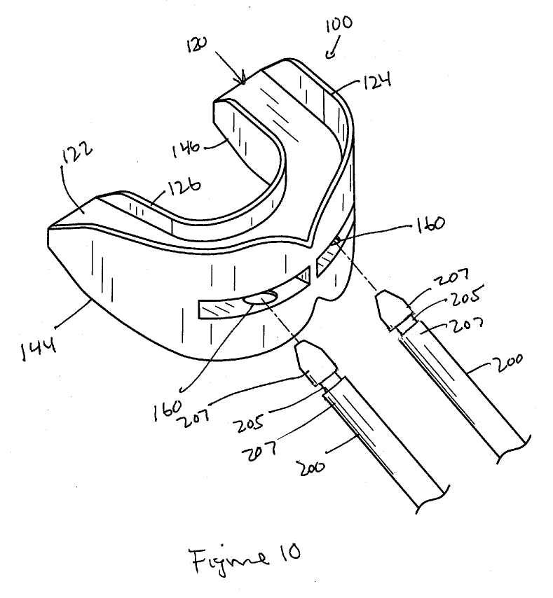

In another embodiment, FIGS. 5A through 9, there is provided a mouthpiece 100 that is easily accommodating in a patient's mouth and can be easily equipped with a UV-light delivery system. The mouthpiece 100 may generally be defined as an U-shaped double trough mouth piece integrally molded as a unitary piece. The double trough mouthpiece 100 comprises an upper trough 120 and a lower trough 140 (shown in FIGS. 7A-7C) interconnected by a relatively flat U-shaped base surface 105. The base surface 105 provides an upper base surface 122 and a lower base surface 142 adapted to engage the upper teeth and lower teeth respectively in use.

The upper trough 120 is defined by an outer peripheral wall member or flange 124 and a laterally spaced smaller inner peripheral wall member or flange 126 both of which interconnect with the upper base surface 122. The outer peripheral wall member 124 has a defined height adapted to cover the buccal side of the upper teeth and may extend such that it covers part of the upper gums. The outer peripheral wall 124 further includes an indent 128 located midway of the circular intermediate section 130 opposite the open end 132 of the U-shaped mouth piece where the indent 128 accommodates tissues (labial frenulum) on the front buccal side of the upper jaw. The inner peripheral wall 126 is relatively smaller in height than the height of the outer peripheral wall 124 and adapted to cover and protect the lingual side of the upper teeth. Both the outer peripheral wall member 124 and inner peripheral wall member 126 are resilient and preferably rather flexible to provide a conforming comfortable fit with the upper teeth.

The lower trough 140 of the mouth piece 100 is similarly structured and defined by an outer peripheral wall member or flange 144 and a laterally spaced smaller inner peripheral wall member or flange 146 both of which interconnect with the lower base surface 142. The outer peripheral wall member 144 has a defined height adapted to cover the buccal side of the lower teeth and may extend such that it covers part of the lower gums. The outer peripheral wall 144 further includes an indent 148 located midway of the circular intermediate section 130 opposite the open end 132 of the U-shaped mouth piece where the indent 148 accommodates tissues on the front buccal side of the lower jaw. The inner peripheral wall 146 is relatively smaller in height than the height of the outer peripheral wall 144 and adapted to cover and protect the lingual side of the lower teeth. Both the outer peripheral wall member 144 and inner peripheral wall member 146 are resilient and preferably rather flexible to provide a conforming comfortable fit with the lower teeth.

The mouth piece 100 includes a pair of windowed apertures 152 centrally located in a middle region 150 of the circular intermediate section 130. The windowed apertures 152 bore through the mouth piece 100 from a front portion 154 of the combined upper outer peripheral wall 124 and the lower outer peripheral wall 144 to a rear portion 156 of the combined upper inner peripheral wall 126 and the lower inner peripheral wall 146. The windowed apertures further define a lower sill section 158 traversing from the front to real portions of the mouth piece 100. A pair of openings 160 are positioned through the lower sill section 158 and out the lower base surface 142 of the U-shaped base surface 105. The openings are sized to accommodate a tubing 54 which includes the source of UV-light.

Once the tubing 54 are inserted through the openings 160, the tips 30 of the tubing extend out the bottom of the mouth piece attempting to make sure the UV-light is properly directed to the mucus membrane under the tongue.

Referring now to 10, in another embodiment the mouth piece 100 is used in connection with tubing 200 (that while is similar in function to the above tubing 54), which further includes indented regions 205 near the tips 30. The indented regions 205 are sized to fit in the openings 160 defined in the mouth piece 100, such that the undented regions 207 on either side of the indented regions 205 secure the tubing 200 in place. Thereby ensuring the tubing 200 cannot be inserted to far into a person's mouth. It being further understood that the tubing could include an enlarged region passed the tip along the tubing such that the tubing inserted into an opening 160 would stop when the enlarged region came up against the opening 160. Being enlarged such that it could not fit through the opening 160.

From the foregoing and as mentioned above, it will be observed that numerous variations and modifications may be effected without departing from the spirit and scope of the novel concept of the invention. It is to be understood that no limitation with respect to the specific methods and apparatus illustrated herein is intended or should be inferred.

I claim:

1. A system for delivering UV-light under a patient's tongue, comprising:
a UV-light source in communication with a pair of tubes for transmitting the UV-light from the source to end tips defined by the pair of tubes separately; and
a mouth piece comprising:
a U-shaped double trough region defined as an upper trough and a lower trough adapted to engage upper and lower teeth of a patient, respectively, the upper trough and lower trough being interconnected by a relatively flat base surface, the base surface provides for an upper base surface for the upper trough and a lower base surface for the lower trough,
the upper trough being defined by an upper outer peripheral wall member and an upper inner peripheral wall member laterally spaced from the upper outer peripheral wall member, both the upper outer and upper inner peripheral wall members being interconnected with the upper base surface,
the lower trough being defined by a lower outer peripheral wall member and a lower inner peripheral wall member laterally spaced from the lower outer peripheral wall member, both the lower outer and lower inner peripheral wall members being interconnected with the lower base surface, and
a pair of windowed apertures centrally located in the double trough region and extending from a front portion to a rear portion of the double trough region, each windowed aperture includes a pair of openings extending out through the lower base surface, the pair of openings being sized to accommodate the pair of tubes such that the tips of the tubes are capable of extending through the openings and exiting through the lower base surface.

2. The system of claim 1, wherein the upper outer peripheral wall member having a defined height higher than a defined height of the upper inner peripheral wall member.

3. The system of claim 2, wherein the upper outer peripheral wall member further includes an indent positioned midway of the U-shaped upper trough.

4. The system of claim 2, wherein the lower outer peripheral wall member further includes an indent positioned midway of the U-shaped lower trough.

5. The system of claim 1, wherein the front portion of the double trough region is defined by the combined upper outer and lower outer peripheral wall members

6. The system of claim 1, wherein the rear portion of the double trough region is defined by the combined upper inner and lower inner peripheral wall members

7. The system of claim 1, wherein the pair of tubes further includes an indented region sized to fit within the pair of openings in the windowed apertures.

8. The system of claim 1, wherein the pair of tubes includes an enlarged region at a distance away from the tip, the enlarged region having a diameter larger then a diameter of an opening from the pair of openings, such that the tubes when inserted through the openings extend through the opening until the enlarged region rests against the opening.

9. A system for delivering UV-light under a patient's tongue, comprising:
a UV-light source in communication with a pair of tubes for transmitting the UV-light from the source to end tips defined by the pair of tubes separately; and
a mouth piece comprising:
a U-shaped double trough region defined as an upper trough and a lower trough adapted to engage upper and lower teeth of a patient, respectively, the upper trough and lower trough being interconnected by a relatively flat base surface, the base surface provides for an upper base surface for the upper trough and a lower base surface for the lower trough, and
a pair of windowed apertures centrally located in the double trough region and extending from a front portion to a rear portion of the double trough region, each windowed aperture includes a pair of openings extending out through the lower base surface, the pair of openings being sized to accommodate the pair of tubes such that the tips of the tubes are capable of extending through the openings and exiting through the lower base surface.

10. The system of claim 9, wherein the upper trough being defined by an upper outer peripheral wall member and an upper inner peripheral wall member laterally spaced from the upper outer peripheral wall member, both the upper outer and upper inner peripheral wall members being interconnected with the upper base surface.

11. The system of claim 9, wherein the lower trough being defined by a lower outer peripheral wall member and a lower inner peripheral wall member laterally spaced from the lower outer peripheral wall member, both the lower outer and lower inner peripheral wall members being interconnected with the lower base surface.

12. The system of claim 10, wherein the upper outer peripheral wall member further includes an indent positioned midway of the U-shaped upper trough.

13. The system of claim 11, wherein the lower outer peripheral wall member further includes an indent positioned midway of the U-shaped lower trough.

14. The system of claim 9, wherein the pair of tubes further includes an indented region sized to fit within the pair of openings in the windowed apertures.

15. The system of claim 9, wherein the pair of tubes includes an enlarged region at a distance away from the tip, the enlarged region having a diameter larger then a diameter of an

16. A blood illuminator comprising:
a light source adapted to emit UV-C light and a power supply for supplying power to the light source;
a pair of light guides, each light guide having a first end for receiving the UV-C light from the light source and having a second end adapted to emit the UV-C light;
a mouth piece having a pair of bored apertures separately corresponding to the pair of light guides, each bored apertures having an entrance near a front portion of said mouth piece and an exit near a bottom portion of said mouth piece, wherein the pair of light guides are inserted through the pair of board apertures, such that the second ends of the pair of light guides may extend outwardly from the bottom portion of said mouthpiece.

17. The illuminator of claim 16, wherein the light guide has a liquid core for transporting the UV-C light from the first end to the second end.

18. The illuminator of claim 17, wherein the mouth piece includes a grove along a center region of the front portion.

19. A blood illuminator comprising:
a light source adapted to emit UV-C light and a power supply for supplying power to the light source;
a pair of light guides, each light guide having a first end for receiving the UV-C light from the light source and having a second end adapted to emit the UV-C light;
a mouth piece having a pair of bored apertures separately corresponding to the pair of light guides, each bored apertures having an entrance near a front portion of said mouth piece and an exit near a bottom portion of said mouthpiece, wherein the pair of light guides are inserted through the pair of board apertures, such that the second ends of the pair of light guides may extend outwardly from the bottom portion of said mouth piece and wherein the mouth piece includes a grove along a center region of the front portion.

20. The illuminator of claim 19, wherein the light guide has a liquid core for transporting the UV-C light from the first end to the second end.

21. A method of illuminating blood with light comprising:
providing a light source adapted to emit UV-C light;
providing a pair of light guides, each light guide having a first end for receiving the UV-C light from the light source and having a second end adapted to emit the UV-C light;
placing a mouthpiece in a mouth of a patient, the mouthpiece having a pair of bored apertures separately corresponding to the pair of light guides, each bored apertures having an entrance near a front portion of said mouthpiece and an exit near a bottom portion of said mouthpiece, wherein the pair of light guides are inserted through the pair of board apertures, such that the second ends of the pair of light guides may extend outwardly from the bottom portion of said mouthpiece and towards a bottom region of the mouth of the patient, and wherein the mouthpiece includes a grove along a center region of the front portion to accommodate a labial frenulum defined in the mouth of the patient; and
supplying power to the light source such that the bottom region of the mouth of the patient is illuminated, wherein the UV-C light penetrates capillaries in the mouth to allow the UV-C light to illuminate blood of the patient.
